(19) United States
(12) Patent Application Publication
(10) Pub. No.: US 2025/0186067 A1
(43) Pub. Date: Jun. 12, 2025

(54) CLOG CLEARANCE DEVICE FOR THROMBECTOMY CATHETER

(71) Applicant: WALK VASCULAR, LLC, Irvine, CA (US)

(72) Inventor: Matthew J. Gillick, Murrieta, CA (US)

(21) Appl. No.: 18/966,629

(22) Filed: Dec. 3, 2024

Related U.S. Application Data
(60) Provisional application No. 63/607,746, filed on Dec. 8, 2023.

(51) Int. Cl. A61B 17/22; A61B 17/00; A61B 17/3203; A61B 90/00

(52) U.S. Cl. CPC A61B 17/22; A61B 17/32037; A61B 2017/00398; A61B 2017/22079; A61B 2090/064

(57) ABSTRACT

Disclosed embodiments describe systems and methods to aspirate a thrombus. Embodiments include an elongate shaft configured for placement within a blood vessel of a subject and an aspiration lumen extending along the elongate shaft. Additionally, disclosed embodiments include a roller and a tube backer. The roller is located on a first side of the elongate shaft. The tube backer is on a second side of the elongate shaft and the second side of the elongate shaft is opposite of the first side of the elongate shaft. Embodiments also include a rotator. The rotator is configured to rotate the roller causing the roller to roll along the elongate shaft against the tube backer.

### CROSS-REFERENCE TO RELATED APPLICATIONS

This application claims the benefit of and priority to U.S. Provisional Patent Application Ser. No. 63/607,746 filed Dec. 8, 2023, and entitled “CLOG CLEARANCE DEVICE FOR THROMBECTOMY CATHETER,” which application is expressly incorporated herein by reference in its entirety.

### BACKGROUND

### 1. The Field of the Invention

The present disclosure relates to removal of clots in blood vessels. In more detail, embodiments of the invention relate to devices that include a peristaltic pump roller that rotates to break up thrombotic material.

### 2. The Relevant Technology

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away.

All of the devices described above have limitations as a result of individual design characteristics. For example, the various available aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable. Even aspiration catheters that employ a high pressure saline jet to macerate and break up the thrombus can become clogged, e.g., if rate of clot flow through the device is too high, or the clot material becomes entangled with the saline lumen within the aspiration catheter.

Mechanical rotary devices use an auger to grab and carry the thrombus away from the target area. Some create transport force via vacuum bottles while others create differential pressure at the distal tip of the device with the auger acting as a low-pressure pump. These devices typically work slowly and offer the physician no feedback as to when the device should be advanced further into the lesion.

Flushing type aspiration devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure.

Accordingly, there is an ongoing need for improved systems to remove thrombotic material.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

What is needed to address existing clogging issues is a device that provides a temporary, quick pressure pulse to dislodge and/or break up any such clog and then allow aspiration to continue. One aspect of the present disclosure relates to a system for aspirating thrombus, including: an elongate shaft configured for placement within a blood vessel of a subject; an aspiration lumen extending along the elongate shaft; a roller, wherein the roller is located to a first side of the elongate shaft; a tube backer, wherein the tube backer is on a second side of the elongate shaft and the second side of the elongate shaft is opposite of the first side of the elongate shaft; and a rotator, wherein the rotator is configured to rotate the roller causing the roller to roll along and compress the elongate shaft against the tube backer.

Another aspect of the present disclosure relates to a method for aspirating thrombus, including providing a system for aspirating a thrombus. Such a system includes an elongate shaft configured for placement within a blood vessel of a subject, an aspiration lumen extending along the elongate shaft, and a roller, wherein the roller is located to a first side of the elongate shaft. The system also includes a tube backer, where the tube backer is on a second side of the elongate shaft and the second side of the elongate shaft is opposite of the first side of the elongate shaft. A rotator is also included, where the rotator is configured to rotate the roller causing the roller to roll along the elongate shaft against the tube backer. The method further includes rotating the roller, so as to cause the roller to roll along the elongate shaft against the tube backer, breaking up a thrombus located in the aspiration lumen as a result of a pressure pulse created by the rotating roller, and aspirating the thrombus.

In any of the described embodiments, the roller can act as a component of a pump (e.g., a peristaltic pump).

In any of the described embodiments, rotating the roller can create a pressure pulse (e.g., a positive pressure pulse) within the aspiration lumen.

The pressure pulse can be sufficient to loosen clogged clot or thrombus material, but small enough so as not to fully eject the clot or thrombus back into the patient's anatomy. In some embodiments, the applied pressure pulse is from about 0.015 MPa to about 0.70 MPa.

In any of the described embodiments, the rotator can include an arm and a center of rotation.

In any of the described embodiments, the rotator may rotate the roller in either a clockwise direction or a counterclockwise direction. For example, one direction (e.g., counterclockwise direction) may apply a positive pressure pulse to the distal aspiration end of the aspiration catheter (useful in breaking up a thrombus clog), while the other direction (e.g., clockwise direction) may result in suction pulses at the distal aspiration end of the aspiration catheter. Such pulsation of the suction may be useful, e.g., if the saline drive unit (SDU) vacuum source experiences a fault condition that causes a loss of vacuum.

In any of the described embodiments, the rotator can rotate the roller in both a counterclockwise direction and a clockwise direction.

In any of the described embodiments, the roller rolling along the elongate shaft can cause the elongate shaft to compress.

In any of the described embodiments, the tube backer can have a width (e.g., parallel to the longitudinal axis of the aspiration lumen) of about 0.63 cm to about 10 cm.

In any of the described embodiments, the rotator can rotate the roller at a speed so as to provide 6 tube compressions/min to about 300 tube compressions/min.

In any of the described embodiments, the elongate shaft can include one or a plurality of shearing ridges at the distal end of the aspiration lumen, so as to break up and/or shear away peripheral portions of a thrombus clog.

In any of the described embodiments, the shearing ridges can be located at a distal end of the elongate shaft.

In any of the described embodiments, the system can further include a control system, wherein the control system selectively activates the rotator to rotate.

In any of the described embodiments, the control system can activate the rotator in response to detecting a clog in the aspiration lumen.

In any of the described embodiments, the clog may be detected based on at least one of aspiration flow or pressure conditions (e.g., reduced aspiration flow or a change in aspiration pressure associated with clogging at the distal end).

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

A description of various embodiments and features of the invention will be rendered by reference to various representative embodiments thereof illustrated in the appended drawings. It is appreciated that these drawings depict only typical embodiments of the invention and are therefore not to be considered limiting of its scope.

FIG. 1 is a plan view of exemplary disposable components of a system for aspirating thrombus according to an implementation of the present disclosure.

FIG. 2 is a cross-sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a perspective view of an exemplary system for aspirating thrombus using system components such as those of FIG. 1 according to an implementation of the present disclosure.

FIG. 5 is an exploded view of a portion of the system of FIG. 4 according to an implementation of the present disclosure.

FIG. 6 is a perspective view of an exemplary aspiration catheter according to an implementation of the present disclosure.

FIG. 7 is a cross-sectional view of an exemplary aspiration catheter according to an implementation of the present disclosure.

FIG. 8 schematically illustrates the exemplary aspiration catheter within a body lumen aspirating thrombus according to an implementation of the present disclosure.

FIG. 9 illustrates an exemplary system according to the present disclosure with a peristaltic pump roller and peristaltic pump tube backer.

FIGS. 10A-10D illustrate an example of the peristaltic pump roller rotating against the peristaltic pump tube backer, with the Figures showing progressive rotation of the roller, so as to compress the aspiration catheter.

FIG. 11 illustrates an example aspiration catheter with shearing ridges at the distal end, to better break up a clogging thrombus.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to systems and methods for removing thrombotic material or thrombus using a roller that rolls along an elongate shaft (e.g., of the aspiration catheter). The roller is configured to create a pressure pulse that may break, disrupt, or push thrombus that is stuck in the aspiration lumen, aiding in breaking up the clogged thrombus, and allowing aspiration to continue. Additionally, in some embodiments, the elongate shaft includes a plurality of shearing ridges at a distal end of the aspiration lumen to further break apart a thrombus or thrombotic material stuck in the aspiration lumen. Embodiments advantageously reduce and remove thrombotic clogs in the aspiration lumen without further user intervention (e.g., otherwise requiring removal and reinsertion). Additionally, some embodiments allow aspiration to be re-established, or to continue without removing the catheter system even when the aspiration lumen is fully or partially clogged with thrombotic material.

While the present disclosure will describe a particular implementation of a rotating roller that creates a pressure pulse that aids in breaking apart thrombotic material or thrombus located distally in an aspirational lumen, it should be understood that the devices, systems, and method described herein may be applicable in other environments and to other uses. Additionally, elements described in relation to any embodiment depicted and/or described herein may be combinable with elements described in relation to any other embodiment depicted and/or described herein.

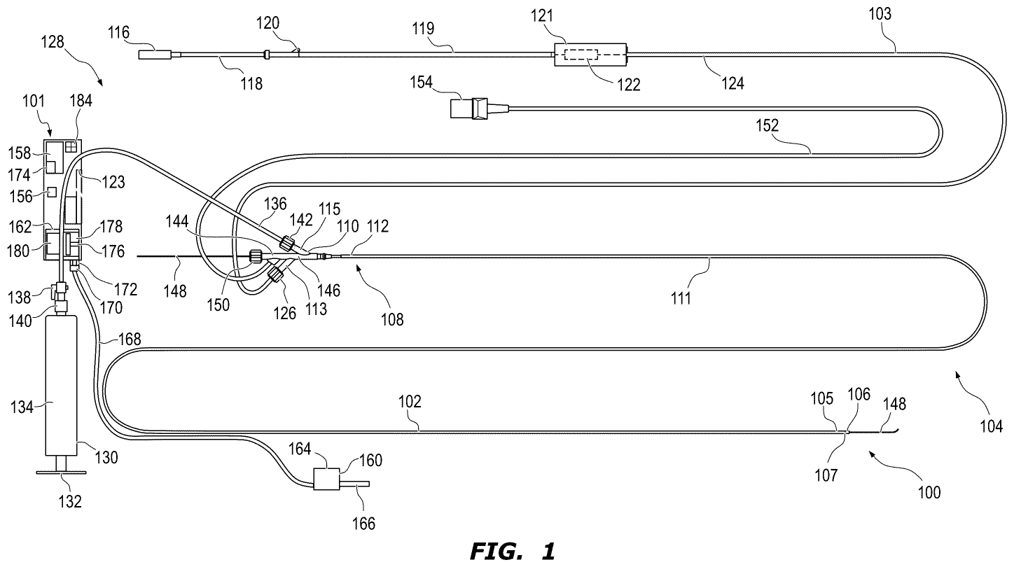

A system 100 for aspirating thrombus is illustrated in FIGS. 1-5, The system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

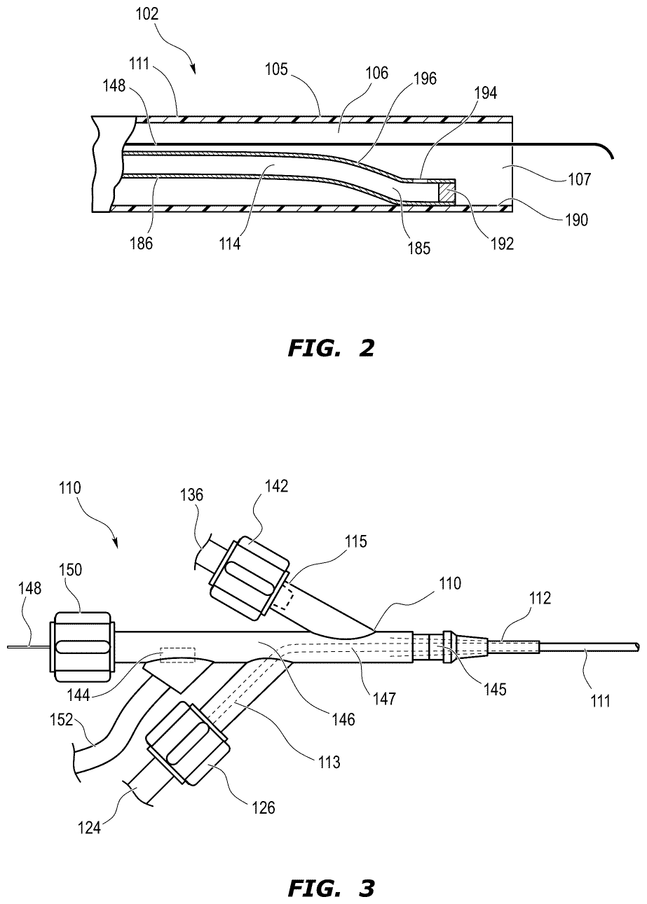

The aspiration catheter 102 has a distal end 105 and includes an over-the-wire guidewire lumen/aspiration lumen 106 extending between an open distal end 107, and a proximal end 108 comprising or connected to a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (not shown) (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector. The connector is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

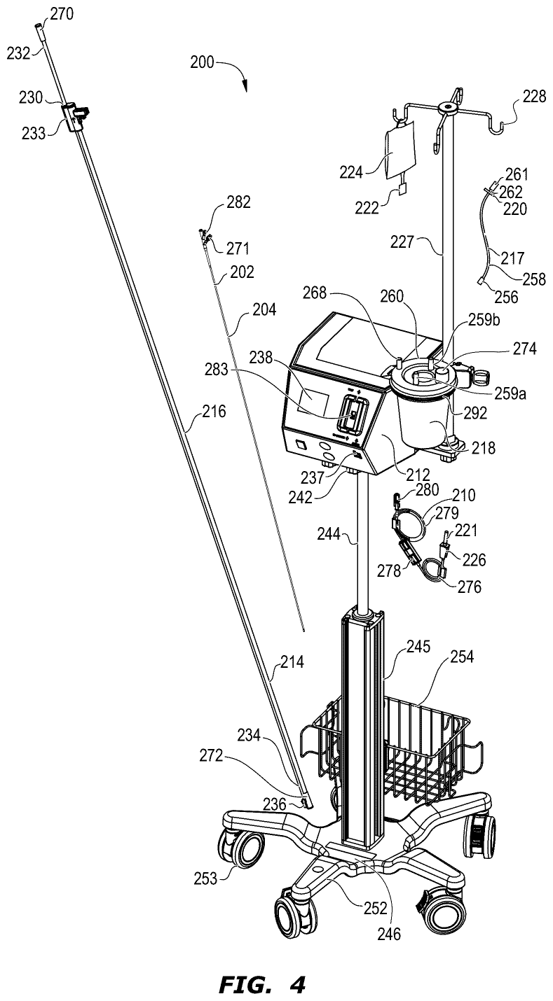

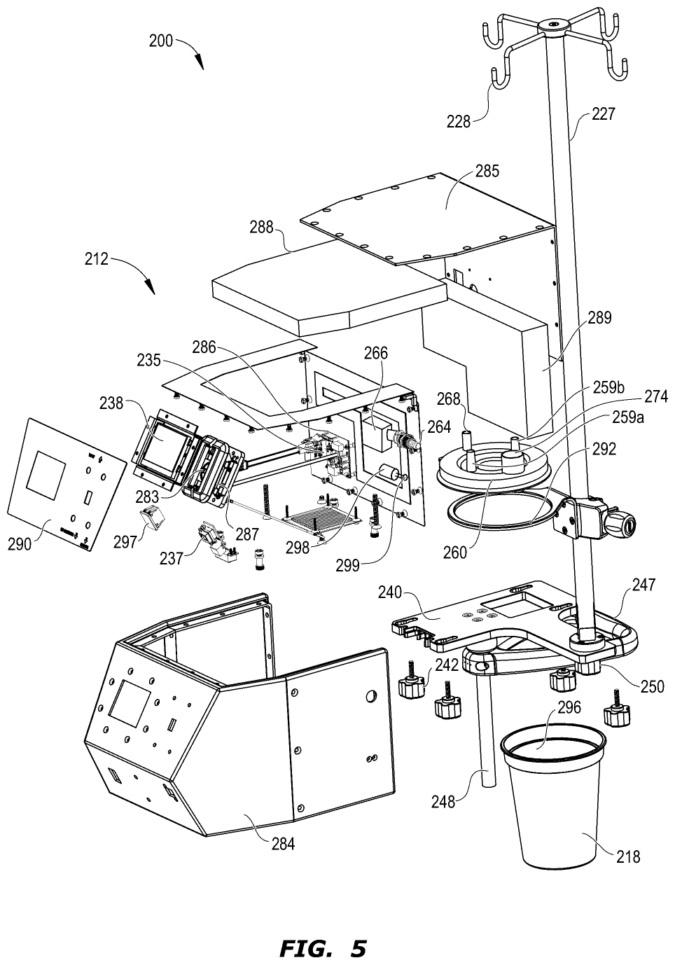

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202 and system 200 may be used in interventional procedures, but may also be used in surgical procedures. The aspiration catheter 202 and system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIGS. 2-3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114 (FIG. 2). In some embodiments, the orifice or opening 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 5 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 278 may couple to the saddle 283 by clipping, friction fitting, vacuum fitting, or other means.

FIG. 5 illustrates embodiments pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 259a and second port 259b for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 259a for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259 for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259b so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

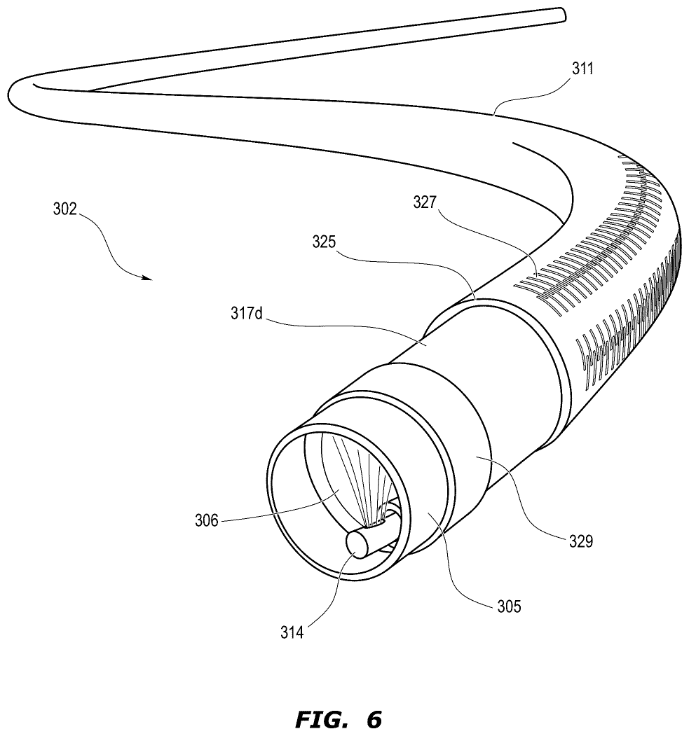

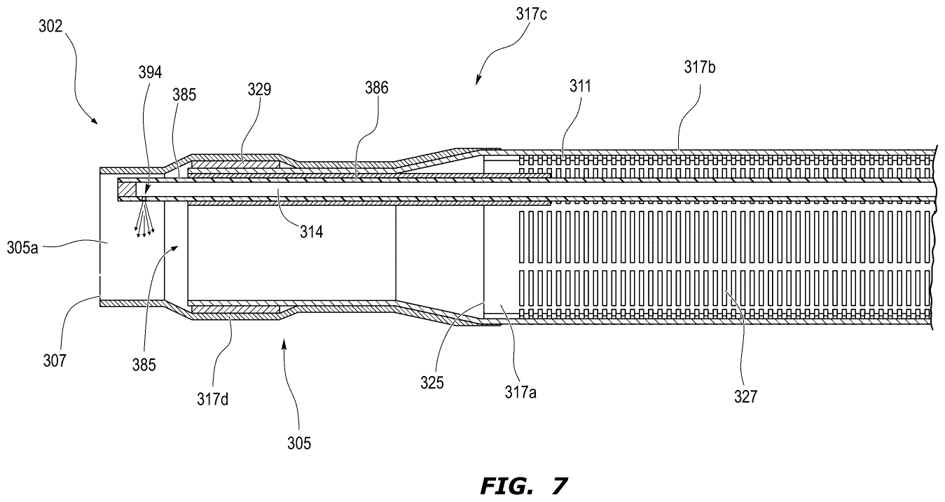

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacketed by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305a or the distal most end of the aspiration catheter with the distal opening 307. The outer jacket 317d protects a distal portion 385 of a supply tube 386 containing a supply lumen 314. While reference is made to a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

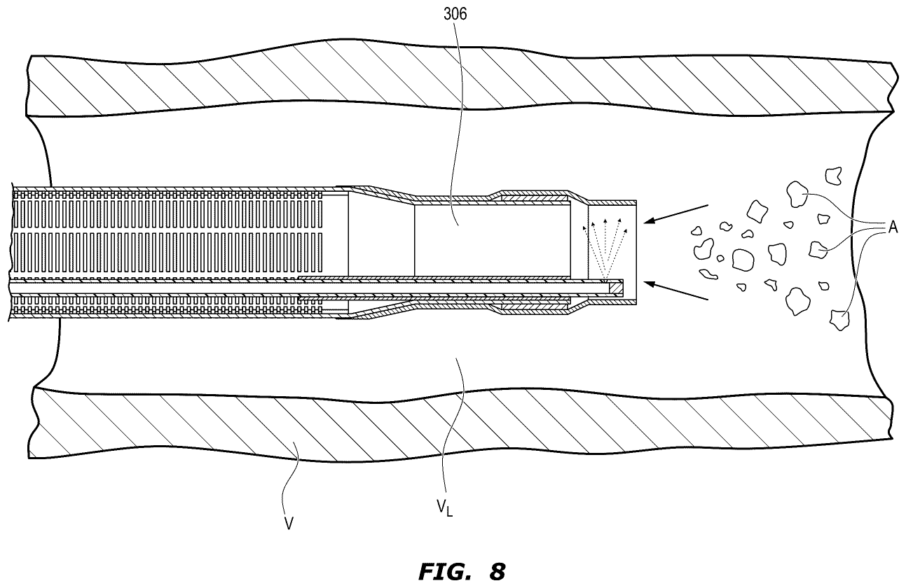

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawn into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of or comprise any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, other elastomer and combinations or modifications thereof.

In some situations, during aspiration, damage to the vessel might occur because the vessel wall is drawn into the aspiration lumen 306 and comes into contact with the jet of pressurized fluid being injected (e.g., a high pressure saline spray at, for example, 650 psi (4.48 Mpa)) from supply lumen 314. By way of example, the supply lumen may provide pressurized fluid (e.g., saline at a pressure from about 3 Mpa to about 6 Mpa). Therefore, a tissue encroachment prevention assembly can prevent aspiration catheter 302 from damaging the vessel during aspiration. The tissue encroachment prevention assembly can be selectively added to the distal end 305 of the aspiration catheter 302 or the distal end 305 can be modified or changed to accommodate the tissue encroachment prevention assembly.

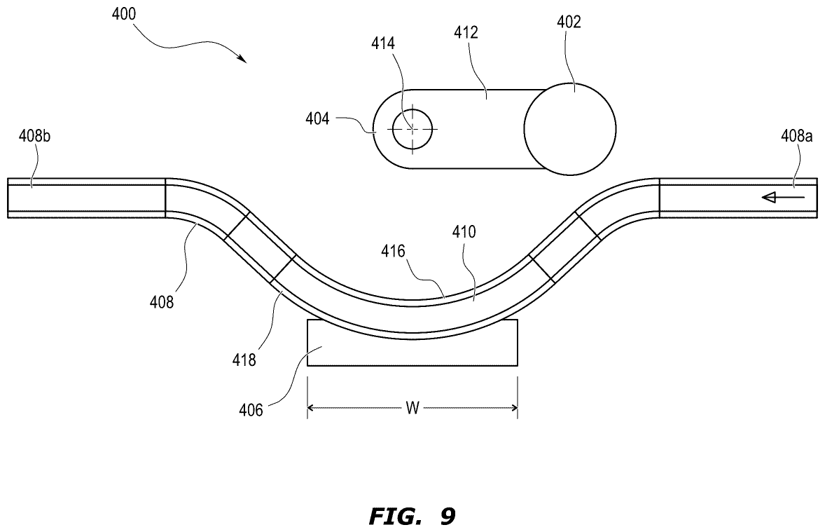

Referring to FIG. 9, the system 400 may be implemented into systems 100, 200 or a similar system, and may include a roller 402 attached to a rotator 404 and a tube backer 406. As shown, the system 400 includes an elongate shaft 408 (e.g., the aspiration catheter of any of the described systems) with an aspiration lumen 410 that extends along the elongate shaft 408. In an embodiment, aspiration flow may pull from the distal end 408a of the elongate shaft 408 towards proximal end 408b of the elongate shaft 408. The proximal end 408b of the elongate shaft 408 may be connected to a suction device (e.g., vacuum pump 266). The roller 402 is located to one side 416 of the elongate shaft 408 while the tube backer is connected to a second side 418 of the elongate shaft 408 that is opposite of the first side 416.

In some embodiments, the roller 402 is a component of a pump such as a peristaltic pump. The roller 402 is attached to a rotator 404. In embodiments, the rotator 404 includes an arm 412 and a center of rotation 414. The arm 412 attaches the roller 402 so as to rotate about the center of rotation 414. The rotator 404 is configured to rotate the roller 402 causing the roller 402 to roll along and compress the elongate shaft 408.

In some embodiments, the rotator 404 rotates the roller 402 in a single direction, such as clockwise or counterclockwise. In embodiments where the rotator 404 rotates the roller 402 in a single direction, the roller 402 may result in a positive pressure pulse within the aspiration lumen, configured to push the clog distally through the aspiration lumen 410. Multiple pulses may be applied, with multiple rotations, by rolling multiple revolutions along the elongate shaft 408. In other embodiments, the rotator 404 rotates the roller 402 in both a clockwise and a counterclockwise direction, e.g., as selected by a user, or automatically selected, according to various sensor data that may be collected during operation. In embodiments where the rotator 404 rotates the roller 402 in multiple directions, a counterclockwise rotation direction may generate a pressure pulse within the aspiration lumen, by pinching the tubing closed (temporarily stopping aspiration), and then pushing the fluid within the suction tubing towards the distal end 408a, resulting in a temporary pressurization of the distal end of the aspiration catheter. Such a pressure pulse may aid in dislodging any clogging thrombus that may be stuck at the distal end of the catheter. A clockwise rotation direction may be useful in temporarily cutting off aspiration at the distal end of the catheter, so as to result in suction pulses. Such may be helpful if the vacuum source of the system experiences a fault condition that results in a loss of vacuum, for example.

When the rotator 404 rolls the roller 402 along the elongate shaft 408, the elongate shaft 408 compresses between the roller 402 and the tube backer 406 which causes a positive, distally directed pressure pulse in the system 400, for a counterclockwise rotation direction. In some embodiments, the rotation creates a pressure pulse of about 0.015 MPa to about 0.70 MPa. The distally directed positive pressure pulse caused by the roller 402 rolling along the elongate shaft 408 and against the tube backer 406 causes the clog to become distally dislodged and/or break apart, and then when aspiration resumes, the broken up thrombus clog material can be aspirated back into the aspiration lumen 410, without further clogging.

In some embodiments, the pressure pulse frequency is a function of the rotor speed of the roller 402 caused by the rotator 404. For example, the rotor speed may be about 6 compressions/minute, about 50 compressions/minute, about 200 compressions/minute, or about 300 compressions/minute. The pulse pressure should be fast enough to have a minimal impact on the aspiration procedure timing. Additionally, in some embodiments, the pressure pulse volume and other characteristics may be adjusted based on the width W of the tube backer 406. The tube backer 406 may be about 0.63 cm, about 2 cm, about 5 cm, or about 10 cm in width. The pulse volume should be large enough to loosen clogged clot material, but small enough so as to not eject the clot fully back into the patient's anatomy.

In some embodiments, the rotator 404 is controlled by a control unit (e.g., any of the control components described in conjunction with FIGS. 1-5). The control unit may activate the rotator 404 to rotate in response to detecting a clog in the aspiration lumen 410 (e.g., a clog at the extreme distal end of the aspiration lumen). The control unit can detect the clog based on aspiration flow, pressure conditions, visual indicators, user input, or other conditions. In some embodiments, the control unit automatically activates the rotator 404 when a clog is detected. In other embodiments, the control unit may notify a user that a clog is detected and further allow the user to activate the rotator 404. Additionally, the control unit may control the speed at which the rotator 404 rotates the roller 402. In some embodiments, the control unit may have a pre-determined roller speed. In other embodiments, the roller speed may be set or changed by a user using the control unit. In yet other embodiments, the control unit may dynamically and automatically change the roller speed, direction, or other characteristics based on detected characteristics of the clog.

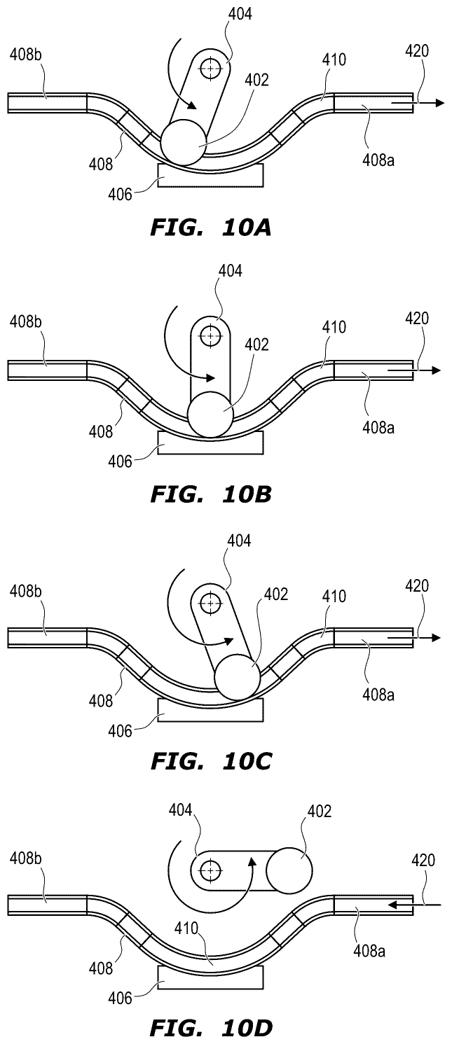

FIGS. 10A-10D illustrate progressive rotation of the roller 402 by the rotator 404 at different points in the pressure pulse cycle. FIG. 10A shows the start of a pressure pulse. As shown, the rotator 404 has been activated to move the roller 402 from the proximal end 408b of the elongate shaft 408 towards the distal end 408a of the elongate shaft 408. In the illustrated configuration, the aspiration lumen 410 has been pinched closed. As shown by the arrow 420, the pressure pulse is distally directed, and causes the flow direction and a pressure pulse that is opposite in direction as compared normal aspiration flow direction shown in FIG. 9. As the roller 402 makes contact with the elongate shaft 408, the elongate shaft 408 is compressed between the roller 402 and the tuber backer 406.

FIG. 10B illustrates the configuration that is present during a mid-stroke of the applied pressure pulse as the roller 402 continues to rotate counterclockwise, against the elongate shaft 404. FIG. 10C illustrates the end of the pressure pulse as the roller 402 completes making contact with the elongate shaft 408, as it rotates counterclockwise.

FIG. 10D illustrates the rotator 404 having rotated the roller 402 further counterclockwise, away from the elongate shaft 408 therefore causing the elongate shaft 408 to no longer be compressed and again opening the aspiration lumen 410. As shown, the flow direction indicated by the arrow 420 is now indicating the restoration of aspiration, with a direction of flow opposite that of the applied pressure pulse. This allows for any broken apart thrombotic material, thrombus, or clog, after having been broken up, to move through the aspiration lumen 410 toward the suction device.

While FIGS. 10A-10D show the roller 402 being rotated by the rotator 404 in a single direction (counterclockwise as shown), in some embodiments, the roller 402 may also be configured to rotate in the opposite direction, e.g., to augment aspiration by providing suction pulses, where aspiration is intermittently cut-off, and restored. Such may be useful, e.g., if the saline drive unit (SDU) vacuum source experiences a fault condition that causes loss of vacuum. Additionally, in some embodiments, the system may roll the roller 402 along the elongate shaft 408 multiple times causing multiple successive pressure pulses until the clog is cleared. In some embodiments, the pressure pulse may vary in each successive roll by changing the roller speed based on detected clog characteristics, or otherwise. For example, when a clog is first detected, the controller may rotate the rotator 404 at a fast rotor speed to push the clog distally, or break apart the detected clog. After the first rotation of the roller 402, the clog may be partially dislodged, and/or broken apart and the controller may decrease the rotor speed of the rotator 404 to further dislodge and/or break apart the clog. This process may occur until the control unit no longer detects a clog (e.g., based on restoration of aspiration pressure when the roller is in a position such as that shown in FIG. 10D, where no occlusion of the aspiration lumen is provided).

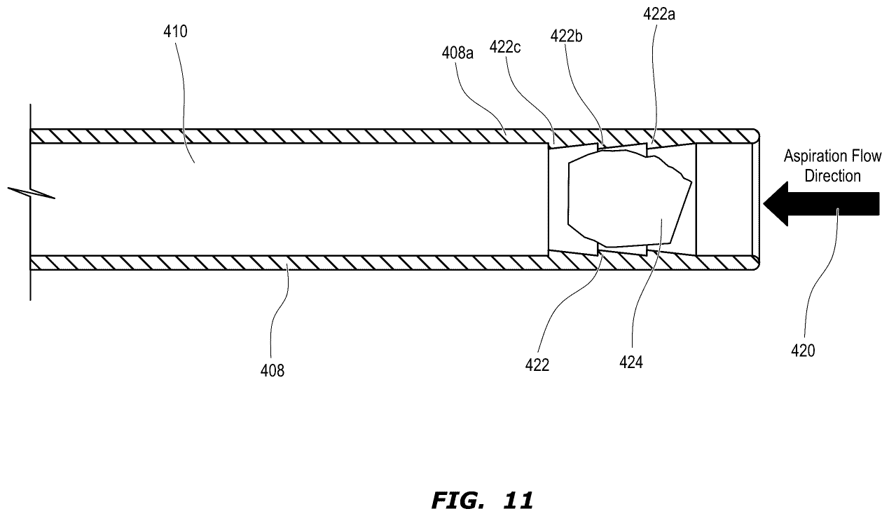

Turning to FIG. 11, in some embodiments, the elongate shaft 408 may further include a plurality of shearing ridges 422 at the distal end of the aspiration lumen (e.g., the extreme distal end thereof, as shown) to further aid in breaking apart the thrombus or thrombotic material 424. The location of such shearing ridges 422 may be proximal to the location of any provided high pressure saline jet, such as that shown in any of FIGS. 6-8. As shown in FIG. 11, as the clog moves proximally in the direction of aspiration flow in the aspiration lumen 410, the shearing ridges make contact with the clog material. The shearing ridges 422 are configured to further break apart or minimize the size of the clog as the clog moves through the aspiration lumen 410. The ridges 422 may be sharp, and biased in a manner that encourages free flow of the clot material 424 in the proximal direction during aspiration, but to impede free movement of the clot 424 in the distal direction. In the event that clot 424 becomes lodged or stuck in this area of the catheter, a pressure pulse from the described system will cause the peripheral portion of the clog to be sheared off, when the clot is forcibly pushed distally (by the applied pressure pulse) against the shearing ridges. When aspiration resumes, the clog with then be smaller, and more easily aspirated through the catheter as a result of clot size reduction due to such shearing action.

In some embodiments, the shearing ridges 422 are located at the distal end 408a of the elongate shaft 408. In other embodiments, the shearing ridges 422 could be located at multiple regions along the elongate shaft 408. In some embodiments, the shearing ridges 422 are located in the elongate shaft 408 distal to the 402, although other placements may also be possible.

As shown in FIG. 11, the illustrated plurality of shearing ridges 422 includes three defined shearing ridges. Such ridges project internally, into lumen 410, resulting in a reduced cross-section within lumen 410, where such ridges are located. The ridges are shown as creating a lumen geometry that is angled or tapered at the location of such ridges, so that the lumen cross-sectional area or diameter is reduced at the location of such projection into the lumen. The lumen geometry is shown as a truncated tapered conical geometry, being narrower proximally, and wider distally. As the clot 424 is pulled proximally into such geometry, is becomes lodged in the decreased cross-sectional portion of the lumen, and then as a pressure pulse is applied, the clot is pushed distally, where contact between the internal ridges and the peripheral portion of the clot causes peripheral portions of the clot to be broken off, reducing the clot size.

While illustrated with three internally projecting ridges, in some embodiments, the plurality of shearing ridges 422 may include one, two, three, four, ten, twenty, or more than twenty shearing ridges 422. A range of values is possible between any of such number of ridges (e.g., 1-20, 2-10, 3-10 ridges, etc.). In some embodiments, the shearing ridges 422 are equally spaced apart, while in other embodiments the shearing ridges 422 may be unequally spaced apart from one another. Additionally, in some embodiments, the shearing ridges 422 may differ in size (e.g., how far they project into lumen 410). For example, the first shearing ridge 422a may be smaller allowing a clog to pass through more easily while the last shearing ridge 422c may be larger to break apart the clog even further as it passes through the aspiration lumen 410. In an embodiment, middle shearing ridge 422b could be of intermediate size (projecting more into lumen 410 than ridge 422a, but less than ridge 422c)

While principally described in the context of use while delivering a jet of saline, it will be appreciated that the presently described systems and methods could be used while delivering other pressurized fluids (e.g., contrast media, or other).

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. A system for aspirating thrombus, comprising: an elongate shaft configured for placement within a blood vessel of a subject; an aspiration lumen extending along the elongate shaft; a roller, wherein the roller is located to a first side of the elongate shaft; a tube backer, wherein the tube backer is on a second side of the elongate shaft and the second side of the elongate shaft is opposite of the first side of the elongate shaft; and a rotator, wherein the rotator is configured to rotate the roller causing the roller to roll along the elongate shaft against the tube backer.

Embodiment 2. The system of embodiment 1, wherein the roller is a component of a pump.

Embodiment 3. The system of any of embodiments 1-2, wherein the pump is a peristaltic pump.

Embodiment 4. The system of any of embodiments 1-3, wherein rotating the roller creates a pressure pulse.

Embodiment 5. The system of any of embodiments 1-4, wherein the pressure pulse is from about 0.015 MPa to about 0.7 MPa.

Embodiment 6. The system of any of embodiments 1-5, wherein the rotator includes an arm and a center of rotation.

Embodiment 7. The system of any of embodiments 1-6, wherein the rotator rotates the roller in either a clockwise direction or a counterclockwise direction.

Embodiment 8. The system of any of embodiments 1-7, wherein the rotator rotates the roller in both a counterclockwise direction and a clockwise direction.

Embodiment 9. The system of any of embodiments 1-8, wherein the roller rolling along the elongate shaft causes the elongate shaft to compress.

Embodiment 10. The system of any of embodiments 1-9, wherein the tube backer has a width from about 0.63 cm to about 10 cm.

Embodiment 11. The system of any of embodiments 1-10, wherein the rotator rotates the roller at a speed so as to provide about 6 compressions/min to about 300 compressions/min.

Embodiment 12. The system of any of embodiments 1-11, wherein the elongate shaft includes a plurality of shearing ridges.

Embodiment 13. The system of any of embodiments 1-12, wherein the plurality of shearing ridges are located at a distal end of the elongate shaft.

Embodiment 14. The system of any of embodiments 1-13, further comprising a control system, wherein the control system activates the rotator to rotate.

Embodiment 15. The system of any of embodiments 1-14, wherein the control system activates the rotator in response to detecting a clog in the aspiration lumen.

Embodiment 16. The system of any of embodiments 1-15, wherein the clog is detected based on at least one of aspiration flow or pressure conditions.

Embodiment 17. A method for aspirating thrombus, comprising: providing a system for aspirating a thrombus, the system comprising: an elongate shaft configured for placement within a blood vessel of a subject; an aspiration lumen extending along the elongate shaft; a roller, wherein the roller is located to a first side of the elongate shaft; a tube backer, wherein the tube backer is on a second side of the elongate shaft and the second side of the elongate shaft is opposite of the first side of the elongate shaft; and a rotator, wherein the rotator is configured to rotate the roller causing the roller to roll along the elongate shaft against the tube backer; rotating the roller, by the rotator, causing the roller to roll along the elongate shaft against the tube backer; breaking up a thrombus located in the aspiration lumen as a result of a pressure pulse created by the rotating roller, and aspirating the thrombus.

Embodiment 18. The method of embodiment 17, wherein the roller is a component of a pump.

Embodiment 19. The method of any of embodiments 17-18, wherein the pump is a peristaltic pump.

Embodiment 20. The method of any of embodiments 17-19, wherein rotating the roller creates a pressure pulse.

Embodiment 21. The method of any of embodiments 17-20, wherein the pressure pulse is from about 0.015 MPa to about 0.70 MPa.

Embodiment 22. The method of any of embodiments 17-21, wherein the rotator includes an arm and a center of rotation.

Embodiment 23. The method of any of embodiments 17-22, wherein rotating the roller occurs in either a clockwise direction or a counterclockwise direction.

Embodiment 24. The method of any of embodiments 17-23, wherein rotating the roller occurs in both a counterclockwise direction and a clockwise direction.

Embodiment 25. The method of any of embodiments 17-24, wherein the roller rolling along the elongate shaft causes the elongate shaft to compress.

Embodiment 26. The method of any of embodiments 17-25, wherein the tube backer has a width from about 0.63 cm to about 10 cm.

Embodiment 27. The method of any of embodiments 17-26, wherein the rotator rotates the roller at a speed so as to provide about 6 compressions/min to about 300 compressions/min.

Embodiment 28. The method of any of embodiments 17-27, wherein the elongate shaft includes a plurality of shearing ridges.

Embodiment 29. The method of any of embodiments 17-28, wherein the plurality of shearing ridges are located at a distal end of the elongate shaft.

Embodiment 30. The method of any of embodiments 17-29, further comprising detecting a clog in the aspiration lumen.

Embodiment 31. The method of any of embodiments 17-30, the system further comprising a control system, wherein the control system activates the rotator to rotate.

Embodiment 32. The method of any of embodiments 17-31, wherein the clog is detected by the control system.

Embodiment 33. The method of any of embodiments 17-32, wherein the clog is detected based on at least one of aspiration flow or pressure conditions.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for aspirating thrombus, comprising:
an elongate shaft configured for placement within a blood vessel of a subject;
an aspiration lumen extending along the elongate shaft;
a roller, wherein the roller is located to a first side of the elongate shaft;
a tube backer, wherein the tube backer is on a second side of the elongate shaft and the second side of the elongate shaft is opposite of the first side of the elongate shaft; and
a rotator, wherein the rotator is configured to rotate the roller causing the roller to roll along the elongate shaft against the tube backer.

2. The system of claim 1, wherein the roller is a component of a pump.

3. The system of claim 2, wherein the pump is a peristaltic pump.

4. The system of claim 1, wherein rotating the roller creates a pressure pulse.

5. The system of claim 4, wherein the pressure pulse is from about 0.015 MPa to about 0.70 MPa.

6. The system of claim 1, wherein the rotator includes an arm and a center of rotation.

7. The system of claim 1, wherein the rotator rotates the roller in either a clockwise direction or a counterclockwise direction.

8. The system of claim 1, wherein the rotator rotates the roller in both a counterclockwise direction and a clockwise direction.

9. The system of claim 1, wherein the roller rolling along the elongate shaft causes the elongate shaft to compress.

10. The system of claim 1, wherein the tube backer has a width from about 0.63 cm to about 10 cm.

11. The system of claim 1, wherein the rotator rotates the roller at a speed so as to provide about 6 compressions/min to about 300 compressions/min.

12. The system of claim 1, wherein the elongate shaft includes a plurality of shearing ridges.

13. The system of claim 12, wherein the plurality of shearing ridges are located at a distal end of the elongate shaft.

14. The system of claim 1, further comprising a control system, wherein the control system activates the rotator to rotate.

15. The system of claim 14, wherein the control system activates the rotator in response to detecting a clog in the aspiration lumen.

16. The system of claim 15, wherein the clog is detected based on at least one of aspiration flow or pressure conditions.

17. A method for aspirating thrombus, comprising:
providing a system for aspirating a thrombus, the system comprising:
an elongate shaft configured for placement within a blood vessel of a subject;
an aspiration lumen extending along the elongate shaft;
a roller, wherein the roller is located to a first side of the elongate shaft;
a tube backer, wherein the tube backer is on a second side of the elongate shaft and the second side of the elongate shaft is opposite of the first side of the elongate shaft; and
a rotator, wherein the rotator is configured to rotate the roller causing the roller to roll along the elongate shaft against the tube backer;
rotating the roller, by the rotator, causing the roller to roll along the elongate shaft against the tube backer;
breaking up a thrombus located in the aspiration lumen as a result of a pressure pulse created by the rotating roller; and
aspirating the macerated thrombus.

18. (canceled)

19. (canceled)

20. (canceled)

21. The method of claim 17, wherein rotating the roller creates a pressure pulse, wherein the pressure pulse is from about 0.015 MPa to about 0.70 MPa.

22. (canceled)

23. The method of claim 17, wherein rotating the roller occurs in either a clockwise direction or a counterclockwise direction.

24. The method of claim 17, wherein rotating the roller occurs in both a counterclockwise direction and a clockwise direction.

25-33. (canceled)
